(19) United States
(12) Patent Application Publication
(10) Pub. No.: US 2014/0277504 A1
(43) Pub. Date: Sep. 18, 2014

(54) Bone Fusion Implant Device

(71) Applicant: OsteoMed LLC, Addison, TX (US)

(72) Inventors: Charles R. Forton, Frisco, TX (US); Seetal Erramilli, Melbourne (AU)

(73) Assignee: OsteoMed LLC, Addison, TX (US)

(21) Appl. No.: 14/213,265

(22) Filed: Mar. 14, 2014

Related U.S. Application Data
(60) Provisional application No. 61/782,036, filed on Mar. 14, 2013.

(51) Int. Cl. A61F 2/44

(52) U.S. Cl. CPC A61F 2/447
USPC 623/17.16

(57) ABSTRACT

Implant devices for insertion between first and second bone structures are disclosed and include an implant device body having an exterior surface and an insertion tool interface including a plurality of apertures configured to secure the implant device to an insertion tool, and includes a first aperture (e.g., a threaded aperture) configured to secure the implant device body to a threaded element of the insertion tool, and a second aperture to receive a finger element of the insertion tool. The plurality of apertures are accessible from at least one edge of the implant device body and extend into the implant device body. The implant device may include a fixation interface including a plurality of screw alignment holes configured to receive and to orient screws at predetermined angles, and to house a head of the screw within the implant device body after insertion of the implant device is complete

### CROSS-REFERENCE TO RELATED APPLICATIONS

The present application claims priority from co-pending U.S. Provisional Patent Application No. 61/782,036, entitled “IMPLANT INSTRUMENTATION INTERCONNECTION,” filed Mar. 14, 2013, and to U.S. patent application Ser. No. 14/041,934, entitled “IMPLANT INSTRUMENTATION INTERCONNECTION,” filed Sep. 30, 2013, the disclosures of which are hereby incorporated herein by reference in their entirety.

### TECHNICAL FIELD

The present application generally relates to implant devices used to stabilize and promote fusion of bone structures.

### BACKGROUND

Intervertebral implant devices are often utilized to promote fusion between two vertebrae. Many different types of surgical tools are utilized to insert these implant devices into a human body. One implant device that generally is placed using an inserter device is a spinal intervertebral spacer. Such a spacer is placed between two vertebrae and used to facilitate fusion of the respective vertebrae. Placing this device requires the use of different forces between a connection point of an insertion tool and the implant device (e.g., pushing, pulling, and prying, etc.), due to the anatomy of the body. Accordingly, an insertion tool and the corresponding implant device must be able to withstand these forces without losing the device or improperly placing the device.

One inserter device which is currently utilized may be referred to as a threaded inserter device. A threaded inserter device generally has a threaded extension that threads onto the implant device to secure the implant device to the insertion tool during insertion and is configured to allow a surgeon to unthread the implant device from the threaded extension after insertion is complete. While such devices have been useful, problems may still occur with exerting force on a connected implant device, rotational forces can be present that can cause the implant device to be misaligned with the insertion space, and in some cases, can cause the implant device to back off of or disengage from the insertion device. Additionally, current threaded insertion devices are not the ideal design to account for torsional or pulling forces. Further, presently available implant devices do not provide solutions to the shortcoming of the presently available insertion tools.

Additionally, techniques presently used to secure or retain the implant devices within the insertion space may make it more difficult to monitor the fusion and may cause rubbing of anatomical features proximate the implant device after insertion. For example, many implant devices require use of a plate that is secured to a first bone structure and a second bone structure. Such plates are typically provided as external components that must be inserted after the insertion of the implant device. In some cases, the plate may be inserted from a different side than the side that the implant device was inserted from. This requires the patient to be moved and/or rotated so that the surgeon can gain access to this different side of the patient and insert the plate. Such movement and rotation may alter the orientation of the implant device within the insertion space. Additionally, insertion of the plate from a different side of the patient may extend a duration of the insertion procedure. Some implant devices have built-in plates designed to retain insertion screws and prevent the screws from backing out of the insertion device. However, images generated by many different imaging systems (e.g., a fluoroscopy imagining system) may be obscured by the metal plate, limiting the ability to monitor the fusion.

### BRIEF SUMMARY

The present disclosure describes various embodiments of an implant device to support and promote bone growth and bone fusion between bone structures, such as vertebrae. It is noted that, although some of the embodiments of an implant device according to the present disclosure are described with reference to supporting and promoting bone growth and bone fusion between vertebrae, one or more of the described embodiments may be used to support and promote bone growth and bone fusion between bone structures other than vertebrae.

An implant device according to one or more embodiments of the present disclosure may include an insertion tool interface configured to secure the implant device to an insertion tool. The insertion tool interface may include two or more apertures configured to interact with components of the insertion tool. A first aperture of the two or more apertures may be a threaded aperture that interacts with a threaded component of the insertion tool and a second aperture of the two or more apertures may be a non-threaded aperture configured to interact with a finger component of the insertion tool. The first aperture may enable the threaded component of the insertion tool to rotatably secure the insertion tool to the implant device and the second aperture may enable the finger of the insertion tool to extend into the aperture and prevent rotation of the implant device relative to the insertion tool during insertion. This may prevent the implant device from disengaging or backing off from the insertion tool and may facilitate more accurate placement of the implant device.

An implant device according to one or more embodiments of the present disclosure may include a fixation interface configured to secure the implant device in a desired orientation within an insertion space between a first bone structure and a second bone structure after insertion of the implant device is complete. The fixation interface may include a plurality of screw alignment holes. Each of the plurality of screw alignment holes may configured to receive a bone screw and orient the screw at a predetermined angle, such that a portion of the screw exits a surface of the implant device and penetrates a bone structure. Each of the plurality of screw alignment holes may define a cavity configured to dispose or embed a head of the screw within the implant device. In some embodiments, such implant devices may not require use of a plate to secure the implant device to one or more bone structures while also providing improved visibility and monitoring of the fusion of the bone structures.

Additionally, an implant device according to one or more embodiments of the present discloser may include one or more screw alignment holes that include locking rings. The locking rings may be configured to prevent the screws from backing out of the implant device or otherwise disengaging the implant device from the insertion space between the first and second bone structures. The locking rings may also prevent rotation (e.g. backing out) of the screws after insertion of the implant device is complete. Such implant devices optionally utilize a plate to secure the implant device to one or more bone structures while also providing improved visibility and monitoring of the fusion of the bone structures

The foregoing has outlined rather broadly the features and technical advantages of the present invention in order that the detailed description that follows may be better understood. Additional features and advantages will be described hereinafter which form the subject of the claims. It should be appreciated by those skilled in the art that the conception and specific embodiment disclosed may be readily utilized as a basis for modifying or designing other structures for carrying out the same purposes of the present application. It should also be realized by those skilled in the art that such equivalent constructions do not depart from the spirit and scope of the application as set forth in the appended claims. The novel features which are believed to be characteristic of embodiments described herein, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present embodiments.

### BRIEF DESCRIPTION OF THE DRAWINGS

For a more complete understanding, reference is now made to the following descriptions taken in conjunction with the accompanying drawings, in which:

FIG. 1 is an illustrative embodiment of an implant device for insertion between a first bone structure and a second bone structure;

FIG. 2 is a second illustrative embodiment of an implant device for insertion between a first bone structure and a second bone structure;

FIG. 3 illustrates a perspective view of the implant device 200 of FIG. 2;

FIG. 4 is an implant device for insertion between a first and second bone structure in accordance with an embodiment of the present application;

FIG. 5 is a second illustration of the implant of FIG. 4;

FIG. 6 is an implant device in accordance with an embodiment of the present application;

FIG. 7 is an illustration of an implant device having an insertion interface and a fixation interface positioned on a lateral edge of the implant device in accordance with an embodiment of the present application;

FIG. 8 is an illustration of a plate that may be used in conjunction an implant device in accordance with one or more embodiments;

FIG. 9 is an illustration of a lateral view of the implant device of FIG. 7 inserted between a first vertebra and a second vertebra;

FIG. 10 is an illustration of an anterior view of the implant device 400 of FIGS. 4 and 5 inserted between a first vertebra and a second vertebra; and

FIG. 11 is an illustration of an anterior view of the implant device of FIG. 1 inserted between a first vertebra and a second vertebra and secured to the first vertebra and the second vertebra using the plate of FIG. 8.

### DETAILED DESCRIPTION

Referring to FIG. 1, an illustrative embodiment of an implant device for insertion between a first bone structure and a second bone structure is shown and designated 100. As shown in FIG. 1, the implant device 100 includes an implant device body that defines a plurality of receptacles 110. The plurality of receptacles 110 includes a first receptacle 112 and a second receptacle 114. The plurality of receptacles 110 may be configured to receive graft material (e.g., material that promotes bone growth) for fusing the first bone structure (e.g., a first vertebra) to the second bone structure (e.g., a second vertebra). Although the implant device body is shown as defining two receptacles (e.g., the first receptacle 112 and the second receptacle 114), it is noted that the implant device body may define a single receptacle, more than two receptacles, or no receptacles in some embodiments.

Additionally, the implant device body has an exterior surface that includes an upper surface 120 that may define a top of the implant device 100 and a lower surface 122 that may define a bottom of the implant device 100. As shown in FIG. 1, the upper surface 120 may include a plurality of ridges 126. The plurality of ridges 126 may be configured to retain the implant device 100 in a desired position and/or orientation between the first bone structure and the second bone structure. For example, the plurality of ridges 126 may be biased or angled in a particular direction to form teeth that may contact and grip a respective bone structure (e.g,. the first bone structure or the second bone structure). Although not visible in FIG. 1, the lower surface 122 of the implant body may include a second plurality of ridges that are substantially similar to the plurality of ridges 126. In an embodiment, the plurality of ridges 126 and/or the plurality of ridges of the lower surface 122 may include a first set of ridges that are biased or angled in a first direction and a second set of ridges that are biased or angled in a second direction. The first set of ridges may include one or more rows of ridges, one or more columns of ridges, a combination of rows and columns of ridges, or another combination of ridges. Ridges of the plurality of ridges that are not included in the first set of ridges may form the second set of ridges. By using different biasing directions or different angle directions, the plurality of ridges may provide better retention of the implant device 100 in the desired position and/or orientation between the first bone structure and the second bone structure.

The implant body has a thickness 124 that may be defined by a distance between the upper surface 120 and the lower surface 122. In an embodiment, the thickness 124 of the implant device body may be uniform (i.e., substantially equal) across the entirety of the implant device body. In an alternative or additional embodiment, the thickness 124 of the implant body may not be uniform. For example, the thickness 124 the implant device body may be thicker along a first edge than a second edge that is adjacent to or that is opposite form the first edge.

For example, in FIG. 1, the one or more edges may include an anterior edge 130, a posterior edge 132, a first lateral edge 134, and a second lateral edge 136. It is noted that although described using edge designators (e.g., anterior, posterior, lateral, etc.), the one or more edges may form a single edge of the implant device body, such as a perimeter edge, and that the edge designators are used for simplicity of description when describing locations and positions of other elements of the implant device 100. Further, use of the edge designators (e.g., anterior, posterior, lateral, etc.) with respect to the edges 130, 132, 134, 136 of the implant device 100 may refer to an orientation of a particular edge of the implant device 100 before, during, or after insertion of the implant device 100. For example, in an embodiment, use of the “anterior” edge designator may indicate that the respective edge (e.g., the anterior edge 130) of the implant device 100 faces an anterior portion (e.g., front) of a patient after insertion of the implant device 100.

In an additional or alternative embodiment, use of a particular edge designator may indicate a direction from which the implant device 100 is inserted between the first bone structure and the second bone structure. For example, when the implant device 100 is inserted from a lateral direction (e.g,. from the side), a lateral edge (e.g., the lateral edge 134 or the lateral edge 136) may be an edge of the implant device 100 that, after insertion, faces the direction from which the implant device 100 was inserted.

As shown in FIG. 1, the implant device 100 includes an insertion tool interface 140 including a plurality of apertures configured to secure the implant device 100 to an insertion tool, such as the insertion tool described in co-pending and commonly assigned U.S. patent application Ser. No. 14/041,934. The plurality of apertures may accessible from at least one edge of the one or more edges of the implant device body, and may extend into the implant device body.

For example, in FIG. 1, the plurality of apertures includes a first aperture 142, a second aperture 144, and a third aperture 146. The first aperture 142 may be a threaded aperture configured to rotatably secure the implant device body to a threaded element of the insertion tool. The second aperture 144 and the third aperture 146 may be configured to receive a first finger element and a second finger element of the insertion tool, as described in co-pending and commonly assigned U.S. patent application Ser. No. 14/041,934. In an embodiment, the insertion tool interface 140 may include the first aperture 142 (e.g., the threaded aperture) and the second aperture 144, but may not include the third aperture 146. In another embodiment, the insertion tool interface 140 may include the first aperture 142 (e.g., the threaded aperture), the second aperture 144, the third aperture 146, and one or more additional apertures (not shown in FIG. 1).

The plurality of apertures of the insertion tool interface 140 may be accessible to the insertion tool from the exterior surface of the implant device body, and may extend into the implant device body. For example, as shown in FIG. 1, the insertion tool interface 140 is positioned along the anterior edge 130 and the plurality of apertures of the insertion tool interface 140 extend from the anterior edge 130 towards the posterior edge 142 of the implant device body. In an alternative embodiment, the insertion tool interface 140 may be positioned along a lateral edge (e.g., the first lateral edge 134 or the second lateral edge 136) and the plurality of apertures of the insertion tool interface 140 may extend into the implant device body and away from the lateral edge of the implant device. An illustrative embodiment of an implant device having an insertion tool interface positioned along a lateral edge of an implant device body is described with reference to FIG. 7. It is further noted that embodiments may also place one or more apertures at oblique angles to allow an insertion tool to approach a target area from an oblique angle.

As shown in FIG. 1, the first aperture 142 (e.g., the threaded aperture) may be disposed within the insertion tool interface 140 between the second aperture 144 and the third aperture 146. In an embodiment, the second aperture 144 and the third aperture 146 may extend into the implant device body in a direction that is parallel to a longitudinal axis 148 of the first aperture 142. In an alternative or additional embodiment, the second aperture 144 and the third aperture 146 may extend into the implant device body in a direction that is angled towards the longitudinal axis 148 of the first aperture 142. In another alternative or additional embodiment, the second aperture 144 and the third aperture 146 may extend into the implant device body in a direction that is angled away from the longitudinal axis 148 of the first aperture 142. It is appreciated that apertures disposed at an angle with respect to the longitudinal axis may provide for additional stability and retention of the implant device with an insertion tool.

In yet another alternative or additional embodiment, the second aperture 144 and the third aperture 146 may extend into the implant device body in different directions. For example, the second aperture 144 may extend into the implant device body in first direction (e.g,. the direction that is angled away from the longitudinal axis 148) and the third aperture 146 may extend into the implant device body in second direction that is different from the first direction (e.g., the direction that is parallel to, or angled towards the longitudinal axis 148). As another example, the second aperture 144 and the third aperture 146 may be angled in a direction that is not oriented relative to the longitudinal axis 148 (e.g., a direction oriented towards the upper surface 120 of the implant device body, a direction that is oriented towards the lower surface 122 of the implant device body, or a combination of the upper and lower surface orientations). Other orientations of the second aperture 144 and the third aperture 146 may be used and are not discussed herein for conciseness.

During operation, a physician may secure the implant device 100 to the insertion tool using the first aperture 142 of the insertion tool interface 140 and a threaded portion of the insertion tool. For example, the threaded portion of the insertion tool may be inserted into the first aperture and then rotated into the first aperture 142. Rotating the threaded portion of the insertion tool into the first aperture may establish a first connection (e.g., a threaded connection) between the implant device 100 and the insertion tool. The first connection may prevent the implant device 100 from disengaging the insertion tool as the implant device 100 is inserted between the first bone structure and the second bone structure.

As the implant device 100 is rotatably secured to the implant device 100 (e.g., by rotating the threaded portion of the insertion tool into the first aperture 142), fingers of the insertion tool may extend into the implant device body via the second aperture 144 and the third aperture 146. The fingers, in conjunction with the second aperture 144 and the third aperture 146, may provide a gripping force that prevents rotation of the implant device 100 as the implant device 100 is inserted between the first bone structure and the second bone structure. Additionally, the gripping force may assist in preventing the implant device 100 from disengaging the insertion tool as the implant device 100 is inserted between the first bone structure and the second bone structure. The gripping force provided by the fingers, the second aperture 144, and the third aperture 146 may be considered a second connection between the implant device 100 and the insertion tool. Thus, the implant device 100 is configured to provide a first connection to an insertion tool and a second connection to the insertion tool during insertion of the implant device 100 between the first bone structure and the second bone structure. Additionally, by preventing rotation of the implant device 100, the second connection may provide improved and/or simplified orientation of the implant device 100 between the first bone structure and the second bone structure during insertion.

After insertion of the implant device 100, the plurality of ridges 126 and/or the second plurality of ridges of the lower surface 122, may provide fixation (e.g., via friction forces caused by compression of the first bone structure and/or the second bone structure) of the implant device 100 between the first bone structure and the second bone structure. The fixation provided by the plurality of ridges 126 and/or the second plurality of ridges of the lower surface 122 may prevent the implant device 100 from shifting or backing out from between the first bone structure and the second bone structure. Thus, the implant device 100 may provide fixation without requiring the implant device 100 to be held in place by a plate and screws attached external to the implant device.

Referring to FIG. 2, a second illustrative embodiment of an implant device for insertion between a first bone structure and a second bone structure is shown and designated 200. As shown in FIG. 2, the implant device 200 includes an implant device body that defines a plurality of receptacles 210. The plurality of receptacles 210 may be the same or substantially similar to the plurality of receptacles 110 of FIG. 1 and may include a first receptacle 212 and a second receptacle 214. The plurality of receptacles 210 may be configured to receive graft material (e.g., material that promotes bone growth) for fusing the first bone structure (e.g., a first vertebra) to the second bone structure (e.g., a second vertebra). Although the implant device body is shown as defining two receptacles (e.g., the first receptacle 212 and the second receptacle 214), it is noted that the implant device body may define a single receptacle, more than two receptacles, or no receptacles in some embodiments.

Additionally, the implant device body has an exterior surface that includes an upper surface 220 that may define a top of the implant device 200 and a lower surface 222 that may define a bottom of the implant device 200. As shown in FIG. 2, the upper surface 220 may include a plurality of ridges 226. The plurality of ridges 226 may be substantially similar to or identical to the plurality of ridges 126 of FIG. 1 and may provide fixation (e.g., via friction forces caused by compression of the first bone structure and/or the second bone structure) of the implant device 200 between the first bone structure and the second bone structure. In an embodiment, the lower surface 222 of the implant device body may include a second plurality of ridges.

The implant body has a thickness 224 that may be defined by a distance between the upper surface 220 and the lower surface 222. In an embodiment, the thickness 224 of the implant device body may be uniform (i.e., substantially equal) across the entirety of the implant device body. In an alternative or additional embodiment, the thickness 224 of the implant body may not be uniform. For example, the thickness 224 the implant device body may be thicker along a first edge than a second edge that is adjacent to or that is opposite form the first edge.

In FIG. 2, the one or more edges may include an anterior edge 130, a posterior edge 132, a first lateral edge 234, and a second lateral edge 236. It is noted that although described using edge designators (e.g., anterior, posterior, lateral, etc.), the one or more edges may form a single edge of the implant device body, such as a perimeter edge, and that the edge designators are used for simplicity of description when describing locations and positions of other elements of the implant device 200. Further, use of the edge designators (e.g., anterior, posterior, lateral, etc.) with respect to the edges 230, 232, 234, 236 of the implant device 100 may refer to an orientation of a particular edge of the implant device 200 before, during, or after insertion of the implant device 200. For example, in an embodiment, use of the “anterior” edge designator may indicate that the respective edge (e.g., the anterior edge 230) of the implant device 200 faces an anterior portion (e.g., front) of a patient after insertion of the implant device 200.

In an additional or alternative embodiment, use of a particular edge designator may indicate a direction from which the implant device 200 is inserted between the first bone structure and the second bone structure. For example, when the implant device 200 is inserted from a lateral direction (e.g,. from the side), a lateral edge (e.g., the first lateral edge 234 or the second lateral edge 236) may be an edge of the implant device 200 that, after insertion, faces the direction from which the implant device 200 was inserted.

In FIG. 2, the implant device 200 includes a fixation interface 240. The fixation interface 240 may include a plurality of screw alignment holes, such as a first screw alignment hole 242, a second screw alignment hole 244, and a third screw alignment hole 246. Each of the screw alignment holes may be configured to receive a screw (e.g., a bone screw), and to orient the screw at a predetermined angle. The predetermined angle may be configured to cause a tip of the screw to extend through the upper surface 220 of the implant device body or the lower surface 222 of the implant device body and into a bone structure (not shown in FIG. 2) adjacent to the respective surface (e.g., the upper surface 220 or the lower surface 222) of the implant device body after insertion of the implant device 200 is complete.

To illustrate, and referring to FIG. 3, a perspective view of the implant device 200 of FIG. 2 is shown with screws rotably secured within screw alignment holes 242, 244, 246 of the fixation interface 240. As shown in FIG. 3, the first alignment screw hole 242 may be configured to orient a first screw 250 at a first predetermined angle to cause a tip of the first screw 250 to extend through the lower surface 222 of the implant device body and into the first bone structure (not shown in FIG. 2) after insertion of the implant device 200 is complete. The second alignment screw hole 244 may be configured to orient a second screw 252 at a second predetermined angle to cause a tip of the second screw to extend through the upper surface 220 of the implant device body and into the second bone structure (not shown in FIG. 2) after insertion of the implant device 200 is complete. The third alignment screw hole 246 may be configured to orient a third screw 254 at the first predetermined angle to cause a tip of the third screw 254 to extend through the lower surface 222 of the implant device body and into the first bone structure (not shown in FIG. 2) after insertion of the implant device 200 is complete.

Although FIG. 3 illustrates orientation of two screws at the first predetermined angle and orientation of a single screw at the second predetermined angle, it should be apparent that the plurality of screw alignment holes may be configured to orient a single screw at the first predetermined angle and two screws at the second predetermined angle, or to orient two or more screws at the first predetermined angle and two or more screws at the second predetermined angle, or to orient screws in more than two predetermined angles. Additionally or alternatively, the plurality of screw alignment holes may be configured to provide orientation at more than two predetermined angles or less than two predetermined angles. The angle of the orientation provided by each of the screw alignment holes may be determined based on a size and/or shape of the first bone structure and the second bone structure, accessibility of the implant device 200 once placed into a desired orientation between the first bone structure and the second bone structure, a drill guide to be used during insertion of the implant device 200, a combination of these factors, and/or additional factors.

In an additional or alternative embodiment, the plurality of screw alignment holes may include more than three screw alignment holes or less than three screw alignment holes. The number of screw alignment holes may be determined, at least in part, based on a particular edge on which the fixation interface 240 is disposed. For example, in FIG. 2, the fixation interface 240 is disposed on the anterior edge 230, which may have a larger surface area than the first lateral edge 234 and the second lateral edge 236. Because of the larger surface area present at the anterior edge 230 relative to the first lateral edge 234 and the second lateral edge 236, the fixation interface 240 may include a greater number of screw alignment holes when disposed on the anterior edge 230 than when the fixation interface 240 is disposed on the first lateral edge 234 or the second lateral edge 236. In addition to determining the number of screw alignment holes to be included in the fixation interface 240 based on the particular edge (or surface area of the particular edge), the number of screw alignment holes included in the fixation interface 240 may be determined, at least in part, based on other factors, such as a size and/or shape of the first bone structure and the second bone structure, accessibility of the implant device 200 once inserted into a desired orientation between the first bone structure and the second bone structure, etc. Additionally, although described as being disposed on an edge surface (e.g., the anterior surface 230) of the implant device body, the plurality of screw alignment holes may be disposed on or overlap portions of multiple surfaces and/or edges of the implant device body. For example, as shown in FIGS. 2 and 3, the first screw alignment hole 242 and the third screw alignment hole 246 are disposed on and overlap portions of the anterior edge 230 and the upper surface 220.

Referring back to FIG. 2, each of the plurality of screw alignment holes defines a cavity configured to house a head of the screw within the implant device body after insertion of the implant device 200 is complete. By disposing the heads of the screws within the implant device body after insertion of the implant device 200 is complete, a likelihood that the implant device 200 will cause rubbing of anatomical features (e.g., the aorta) proximate the first bone structure and the second bone structure after the implant device 200 is inserted may be reduced or eliminated.

Additionally, a locking ring, such as the exemplary locking ring 260, may be embedded within the cavity defined by each of the plurality of screw alignment holes. The locking ring may configured to prevent rotation of a respective screw after insertion of the implant device 200 is complete. As shown in the expanded view of the locking ring 260, the locking rings may include threads 262 configured to rotatably receive a screw (e.g., the first screw 250 of FIG. 3). Friction forces and pressure forces created by the tightening of the screw may cause the screw to fuse (e.g., via cold welding) with the locking ring 260. Because the locking rings are embedded within the plurality of screw alignment holes, the fusion of the screws with the respective locking rings may prevent the screws from backing out of the respective screw alignment holes after insertion of the implant device 200 is complete.

Each of the locking rings may include a c-clip that partially surrounds the locking ring. For example, as shown in the expanded view of the locking ring 260, a c-clip 264 may partially surround the locking ring 260. The c-clip 264 may be said to partially surround the locking ring 260 because a gap 266 may be present between a first end 264A of the c-clip 264 and a second end 264B of the c-clip 264. Each of the plurality of screw alignment holes may include a groove (not shown in FIG. 2) that is configured to receive the c-clip 264 during insertion of the implant device 200. For example, because the c-clip 264 only partially surrounds the locking ring 264, as the screws are tightened, the pressure may cause c-clip 264 to compress (i.e., bring the first end 264A and the second end 264B closer together and decreasing a size of the gap 266), enabling the c-clip 264 to enter the groove of the respective screw alignment hole. The c-clip 264 may be biased such that the c-clip 264 expands once the c-clip 264 has entered the groove, “locking” the locking ring 260 in place (i.e., loosening of the screw will not compress the c-clip and permit the c-clip from exiting the groove. Additionally, the groove may include a notch (not shown in FIG. 2) that is configured to enter the gap 266, to prevent turning of the locking ring 260 and the c-clip 264 after insertion of the implant device 200. In other embodiments, the locking ring 260 may not include the c-clip 264. Instead, an outer surface of the locking ring 260 may have a plurality of teeth that grip the implant device body and prevent rotation of the locking ring 260 and/or the screws. The locking ring 260 including the plurality of teeth may be implanted or embedded within the implant device body using injection molding, or another manufacturing process. Other mechanisms for preventing the locking ring 260 from turning or dislodging from the implant device body may be used and are not discussed here for conciseness of this disclosure. Thus, the use of the term locking ring in the present disclosure and the appended claims is not to be limited to locking rings including c-clips or teeth unless expressly recited in the claims.

By using the locking ring in combination with the groove and the c-clip (or teeth), the implant device 200 may not require use of a plate (e.g., a titanium plate or other metallic plate) to secure and retain the implant device 200 between the first bone structure and the second bone structure. Additionally, the locking ring, in combination with the groove and the c-clip (or teeth), may prevent the screws from backing out of the implant device 200 without requiring the use of hubcaps and/or plates. Because the hubcaps and/or plates are not required, bone growth may be more readily visible using fluoroscopy or other imaging techniques. Additionally, use of the locking ring in combination with the groove and the c-clip may also reduce a likelihood that the implant device 200 will cause rubbing of anatomical features (e.g., the aorta) proximate the first bone structure and the second bone structure after the implant device 200 is inserted. Further, insertion of the implant device 200 may be simplified compared to implant devices requiring plates because the insertion procedure may be performed in its entirety from a single insertion orientation (i.e., the patient would not need to be turned over once the implant device 200 is inserted to perform additional procedures, such as inserting a plate or other mechanism). Still further, insertion of the implant device 200 may be simplified compared to implant devices requiring plates because the insertion procedure may be performed without requiring insertion of additional hardware (e.g., plates) after insertion of the implant device 200 is complete. This may cause reduced recovery times for patients and may reduce an amount of time required to for the surgeon to perform the insertion of the implant device 200.

In an embodiment, the implant device 200 may include an insertion tool interface configured to secure the implant device 200 to an insertion tool. The insertion tool interface may be the insertion tool interface 140 of FIG. 1, as described with reference to FIG. 4, or may be a different insertion tool interface (e.g., an insertion tool interface that does not include finger apertures, such as the second aperture 144 and the third aperture 146 of FIG. 1), as described with reference to FIG. 7. In some embodiments, one or more screw alignment holes may also be configured to act as an implant insertion tool interface as described above.

Referring to FIG. 4, an illustration of an implant device that includes the insertion tool interface 140 of FIG. 1 and the that includes the fixation interface 240 of FIG. 2 is shown and designated 400. As shown in FIG. 4, the insertion tool interface 140 and the fixation interface 240 may be disposed on an anterior edge 430 of the implant device 400, and the insertion tool interface 140 may be positioned between the first screw alignment hole 242 and the third screw alignment hole 246 and above the second screw alignment hole 244. It is noted that other configurations and arrangement of the elements of the insertion tool interface 140 and the elements of the fixation interface 240 may be implemented without departing from the scope of the present disclosure. The screw alignment holes of the fixation interface 240 of the implant device 400 may include locking rings 260 that are configured to prevent the screws from backing out of the implant device 400, as described with reference to FIGS. 2 and 3. Thus, the implant device 400 may not require insertion of a plate subsequent to insertion of the implant device 400. This may reduce an amount of time required to perform insertion of the implant device 400 and may also reduce a likelihood that the implant device 400 will cause rubbing against one or more anatomical features (e.g. nerves, circulatory structures, etc.) proximate the first bone structure and the second bone structure after the implant device 400 is inserted.

Referring to FIG. 5, a second illustration of the implant device 400 of FIG. 4 showing insertion of the screws is shown. As shown in FIG. 5, a first screw 502 (e.g., a first bone screw) has been inserted into the first screw alignment hole 242, a second screw 504 (e.g., a second bone screw) has been inserted into the first screw alignment hole 244, and a third screw 506 (e.g., a third bone screw) has been inserted into the first screw alignment hole 246. Each of the screws 502, 504, 506 may generate friction forces and pressure forces during tightening, and the tightening may cause the screws 502, 504, 506 to fuse (e.g., cold weld to the respective locking rings 260. When the locking rings 260 include c-clips (e.g., the c-clip 264 of FIG. 2), the pressure forces generated by the tightening may cause the c-clip to enter a groove within a respective cavity of one or the screw alignment holes 242, 244, 246.

As shown in FIG. 5, heads of the screws may disposed or seated entirely within the cavities defined by the screw alignment holes 242, 244, 246. For example, after insertion of the implant device 400, a head 502A of the first screw 502 may be disposed or seated entirely within the cavity defined by the first screw alignment hole 242, a head 504A of the second screw 504 may be disposed or seated entirely within the cavity defined by the second screw alignment hole 244, and a head 506A of the third screw 506 may be disposed or seated entirely within the cavity defined by the third screw alignment hole 246. Thus, the implant device 400 may not require insertion of a plate subsequent to insertion of the implant device 400. This may reduce an amount of time required to perform insertion of the implant device 400 and may also reduce a likelihood that the implant device 400 will cause rubbing of anatomical features (e.g., the aorta) proximate the first bone structure and the second bone structure after the implant device 400 is inserted.

Referring to FIG. 6, another embodiment of an implant device that includes an insertion tool interface (such as interface 140 of FIG. 1) and a fixation interface is shown and designated 600. As shown in FIG. 6, the implant device 600 includes the fixation interface 140 of FIG. 1 and a fixation interface that includes a first screw alignment hole 602 and a second screw alignment hole 604. The first screw alignment hole 602 may include a first locking ring (not shown) and may be configured to receive a first screw 610 (e.g., a first bone screw) and the second screw alignment hole 604 may include a second locking ring (not shown) and may be configured to receive a second screw 612 (e.g., a first bone screw). As shown in FIG. 6, the fixation interface and the insertion tool interface 140 may be positioned on an anterior edge 630 of the implant device 600, and the insertion tool interface 140 may be positioned on the anterior edge 630 between the first screw alignment hole 602 and the second screw alignment hole 604.

When the fixation interface includes two screw alignment holes, the two screw alignment holes may provide different orientations for the respective screws. For example, in FIG. 6, the first screw alignment hole 602 may be configured to orient the first screw 610 at a first predetermined angle that causes a tip of the first screw 610 to extend beyond an upper surface 620 of the implant device 600 and into a first bone structure (not shown) adjacent to the upper surface 620 of the implant device 600. The second screw alignment hole 604 may be configured to orient the second screw 612 at a second predetermined angle that causes a tip of the second screw 612 to extend beyond a lower surface 622 of the implant device 600 and into a second bone structure (not shown) adjacent to the lower surface 622 of the implant device 600.

The implant device 600 may include locking rings (e.g., the locking rings 260 of FIG. 2) that are disposed or embedded within the cavities defined by the first screw alignment hole 602 and the second screw alignment hole 604 of the implant device 600. Additionally, respective heads of the first screw 610 and the second screw 612 may be disposed or seated entirely within the cavities defined by the first screw alignment hole 602 and the second screw alignment hole 604. Thus, the implant device 600 may not require insertion of a plate subsequent to insertion of the implant device 600. This may reduce an amount of time required to perform insertion of the implant device 600 and may also reduce a likelihood that the implant device 600 will cause rubbing of anatomical features (e.g., the aorta) proximate the first bone structure and the second bone structure after the implant device 600 is inserted.

Referring to FIG. 7, an illustration of an implant device having an insertion interface and a fixation interface positioned on a lateral edge of the implant device is shown and designated 700. As shown in FIG. 7, the insertion tool interface of the implant device 700 includes an aperture 710. The aperture 710 may be a threaded aperture and may be configured to rotatably secure the implant device 700 to a threaded portion of an insertion tool. Although additional apertures for receiving fingers of an insertion tool are not illustrated in FIG. 7, such an embodiment is within the scope of the present disclosure and is not illustrated herein for conciseness and simplicity of description. The fixation interface of the implant device 700 includes a first screw alignment hole 702 and a second screw alignment hole 704. In some embodiments, one or more of screw alignment holes 702 and 704 may function as apertures for receiving one or more fingers of an inserter tool.

When the fixation interface includes two screw alignment holes, the two screw alignment holes may provide different orientations for the respective screws. For example, in FIG. 7, the first screw alignment hole 702 may be configured to orient a first screw 740 at a first predetermined angle that causes a tip of the first screw 740 to extend beyond an upper surface 720 of the implant device 700 and into a first bone structure (not shown) adjacent to the upper surface 720 of the implant device 700. The second screw alignment hole 704 may be configured to orient a second screw 742 at a second predetermined angle that causes a tip of the second screw 742 to extend beyond a lower surface 722 of the implant device 700 and into a second bone structure (not shown) adjacent to the lower surface 722 of the implant device 700.

The implant device 700 may include locking rings (e.g., the locking rings 260 of FIG. 2) that are disposed or embedded within the cavities defined by the first screw alignment hole 702 and the second screw alignment hole 704 of the implant device 700. Additionally, respective heads of the first screw 740 and the second screw 742 may be disposed or seated entirely within the cavities defined by the first screw alignment hole 702 and the second screw alignment hole 704. Thus, the implant device 700 may not require insertion of a plate subsequent to insertion of the implant device 700. This may reduce an amount of time required to perform insertion of the implant device 700 and may also reduce a likelihood that the implant device 700 will cause rubbing of anatomical features (e.g., the aorta) proximate the first bone structure and the second bone structure after the implant device 700 is inserted.

Referring to FIG. 8, an illustration of a plate that may be used in conjunction with the implant device 100 of FIG. 1 is shown and designated 800. As shown in FIG. 8, the plate 800 may include fixation interface that includes a first screw alignment hole 810, a second screw alignment hole 820, and a third screw alignment hole 830. The first screw alignment hole 810 may be configured to receive a first screw 812 (e.g., a first bone screw or another type of screw) and to orient the first screw 812 into a first bone structure (not shown in FIG. 8) at a first predetermined angle. The second screw alignment hole 820 may be configured to receive a second screw 822 (e.g., a second bone screw or another type of screw) and to orient the second screw 822 into the first aperture 142 (not shown in FIG. 8) along the longitudinal axis 148. The third screw alignment hole 830 may be configured to receive a third screw 832 (e.g., a third bone screw or another type of screw) and to orient the third screw 832 into a second bone structure (not shown in FIG. 8) at a second predetermined angle. Thus, the first screw 812 and the third screw 832 may limit the range of motion of the first bone structure and the second bone structure to provide a desired amount of compression and/or pressure to stimulate and promote bone growth while the second screw 822 may secure the implant device 100 in the desired orientation (e.g., the orientation provided by the interaction of the first aperture 142, the second aperture 144, and the third aperture 146 with the insertion tool, as described with reference to FIG. 1). In an aspect, each of the screw alignment holes 810, 820, 830 may include a locking ring, such as the locking ring 260 of FIG. 2 or another structure configured to perform the function of the locking ring 260 (e.g., preventing the screw from backing out from the respective bone structure and the plate 800), such as a structure configured to facilitate cold welding of the screw to a respective one of the screw alignment holes 810, 820, 830 as the screw is tightened. Respective heads of the screws 812, 822, 832 may be disposed or seated entirely within the cavities defined by the screw alignment holes 810, 820, 830 after being tightened.

Referring to FIG. 9, an illustration of a lateral view of the implant device 700 of FIG. 7 is shown inserted between a first vertebra (e.g., a first bone structure) and a second vertebra (e.g., a second bone structure) is shown. Referring to FIG. 10, an illustration of an anterior view of the implant device 400 of FIGS. 4 and 5 is shown inserted between a first vertebra (e.g., a first bone structure) and a second vertebra (e.g., a second bone structure) is shown. Referring to FIG. 11, an illustration of an anterior view of the implant device 100 of FIG. 1 is shown inserted between a first vertebra (e.g., a first bone structure) and a second vertebra (e.g., a second bone structure) is shown. As shown in FIG. 11, the implant device 100 may be secured to the first vertebra and the second vertebra using the plate 800 of FIG. 8.

One or more of the implant devices, or a component thereof, described with reference to FIGS. 1-11 may be formed using metals (e.g., titanium), polymers, ceramics, glasses, composite materials, biological materials or tissues, insulators, conductors, semiconductors, or other biocompatible or non-biocompatible materials. Different materials may be used for individual components. Different materials may be combined in a single component. In some embodiments, the implant device body may be formed using polyetheretherketone (PEEK), either alone or in combination with other materials. Using PEEK to form the implant device may be beneficial because it approximates a modulus strength of bone. As compressive forces are applied to the bone by the PEEK implant device (or the pressure and friction forces generated by the screws), the bone structures and various tissues and cells react, creating structure fusion mass of bone. Additionally, using PEEK implant devices may cause the fusion mass to take more load faster than with titanium implant devices (i.e., because the titanium implant device does not compress the bones as efficiently as the PEEK implant device).

It should be understood that the present system, kits, apparatuses, and methods are not intended to be limited to the particular forms disclosed. Rather, they are to cover all combinations, modifications, equivalents, and alternatives falling within the scope of the claims.

The claims are not to be interpreted as including means-plus- or step-plus-function limitations, unless such a limitation is explicitly recited in a given claim using the phrase(s) “means for” or “step for,” respectively.

The term “coupled” is defined as connected, although not necessarily directly, and not necessarily mechanically.

The use of the word “a” or “an” when used in conjunction with the term “comprising” in the claims and/or the specification may mean “one,” but it is also consistent with the meaning of “one or more” or “at least one.” The term “about” means, in general, the stated value plus or minus 5%. The use of the term “or” in the claims is used to mean “and/or” unless explicitly indicated to refer to alternatives only or the alternative are mutually exclusive, although the disclosure supports a definition that refers to only alternatives and “and/or.”

The terms “comprise” (and any form of comprise, such as “comprises” and “comprising”), “have” (and any form of have, such as “has” and “having”), “include” (and any form of include, such as “includes” and “including”) and “contain” (and any form of contain, such as “contains” and “containing”) are open-ended linking verbs. As a result, a method or device that “comprises,” “has,” “includes” or “contains” one or more steps or elements, possesses those one or more steps or elements, but is not limited to possessing only those one or more elements. Likewise, a step of a method or an element of a device that “comprises,” “has,” “includes” or “contains” one or more features, possesses those one or more features, but is not limited to possessing only those one or more features. Furthermore, a device or structure that is configured in a certain way is configured in at least that way, but may also be configured in ways that are not listed.

In the foregoing Detailed Description, various features are grouped together in several embodiments for the purpose of streamlining the disclosure. This method of disclosure is not to be interpreted as reflecting an intention that the disclosed embodiments require more features than are expressly recited in each claim. Rather, as the following claims reflect, inventive subject matter may lie in less than all features of a single disclosed embodiment. Thus, the following claims are hereby incorporated into the Detailed Description, with each claim standing on its own as a separate embodiment.

Although the embodiments of the present disclosure and their advantages have been described in detail, it should be understood that various changes, substitutions and alterations can be made herein without departing from the spirit and scope of the disclosure as defined by the appended claims. Moreover, the scope of the present disclosure is not intended to be limited to the particular embodiments of the process, machine, manufacture, composition of matter, means, methods and steps described herein. As one of ordinary skill in the art will readily appreciate from the present disclosure, processes, machines, manufacture, compositions of matter, means, methods, or steps, presently existing or later to be developed that perform substantially the same function or achieve substantially the same result as the corresponding embodiments described herein may be utilized according to the present disclosure. Accordingly, the appended claims are intended to include within their scope such processes, machines, manufacture, compositions of matter, means, methods, or steps.

What is claimed is:

1. An implant device for insertion between a first bone structure and a second bone structure, the implant device comprising:
an implant device body including an exterior surface, wherein the exterior surface of the implant device body includes an upper surface defining a top of the implant device, a lower surface defining a bottom of the implant device, and one or more edges defining a portion of the exterior surface between the upper surface and the lower surface, wherein the implant body has a thickness defined by the upper surface and the lower surface, and wherein the implant body defines a plurality of receptacles configured to receive graft material for fusing the first bone structure to the second bone structure; and
an insertion tool interface including a plurality of apertures configured to secure the implant device to an insertion tool, wherein the plurality of apertures are accessible from at least one edge of the one or more edges of the implant device body and extend into the implant device body, wherein a first aperture of the plurality of apertures is a threaded aperture configured to rotatably secure the implant device body to a threaded element of the insertion tool, and wherein a second aperture of the plurality of apertures is configured to receive a finger element of the insertion tool.

2. The implant device of claim 1, wherein the at least one edge of the implant device body is an anterior edge of the implant device, and wherein the plurality of apertures extend into the implant device body from the anterior edge towards a posterior edge of the implant device.

3. The implant device of claim 1, wherein the at least one edge of the implant device body is a lateral edge of the implant device, and wherein the plurality of apertures extend into the implant device body and away from the lateral edge of the implant device.

4. The implant device of claim 1, wherein the plurality of apertures includes a third aperture configured to receive a second finger element of the insertion tool, wherein the threaded aperture is disposed within the insertion tool interface between the second aperture and the third aperture.

5. The implant device of claim 4, wherein the second aperture and the third aperture extend into the implant device body in a direction that is parallel to a longitudinal axis of the threaded aperture, a direction that is angled towards the longitudinal axis of the threaded aperture, a direction that is angled away from the longitudinal axis of the threaded aperture, or a combination thereof.

5. The implant device of claim 4, wherein the implant device includes a fixation interface includes a plurality of screw alignment holes, wherein each of the plurality of screw alignment holes is configured to receive a screw and to orient the screw at a predetermined angle.

6. The implant device of claim 5, wherein each of the plurality of screw alignment holes defines a cavity configured to house a head of the screw within the implant device body after insertion of the implant device is complete.

7. The implant device of claim 6, wherein the predetermined angle is configured to cause a tip of the screw to extend through the upper surface of the implant device body or the lower surface of the implant device body and into a bone structure adjacent to the implant device body after insertion of the implant device body is complete.

8. The implant device of claim 6, wherein at least one of the plurality of screw alignment holes includes a locking ring configured to prevent rotation of the screw after insertion of the implant device body is complete.

9. The implant device of claim 5, wherein the plurality of screw alignment holes includes a first alignment screw hole configured to orient a first screw at a first predetermined angle to cause a tip of the first screw to extend through the upper surface of the implant device body and into the first bone structure after insertion of the implant device body is complete, and a second alignment screw hole configured to orient a second screw at a second predetermined angle to cause a tip of the second screw to extend through the lower surface of the implant device and into the second bone structure after insertion of the implant device body is complete.

10. The implant device of claim 9, wherein the plurality of screw alignment holes includes a third screw alignment hole configured to orient a third screw at the first predetermined angle or the second predetermined angle to cause a tip of the third screw to extend through the upper surface of the implant device body and into the first bone structure after insertion of the implant device body is complete or to extend through the lower surface of the implant device and into the second bone structure after insertion of the implant device body is complete.

11. The implant device of claim 1, wherein the implant device is secured to the first bone structure and the second bone structure using a plate, wherein the plate includes a plurality of screw alignment holes configured to orient screws into the first bone structure and the second bone structure, and wherein each of the plurality of screw alignment holes includes a locking ring configured to prevent rotation of the screw after insertion of the implant device body is complete.

12. The implant device of claim 11, wherein the plate includes a screw alignment hole configured to orient a screw into the implant device body to secure the implant device in an orientation provided during the insertion of the implant device.

13. The implant device of claim 12, wherein the at least one screw alignment hole orients the screw into the implant device body via the first aperture.

14. An implant device for insertion between a first bone structure and a second bone structure, the implant device comprising:
an implant device body including an exterior surface, wherein the exterior surface of the implant device body includes an upper surface defining a top of the implant device, a lower surface defining a bottom of the implant device, and one or more edges defining a portion of the exterior surface between the upper surface and the lower surface, wherein the implant body has a thickness defined by the upper surface and the lower surface, and wherein the implant body defines a plurality of receptacles configured to receive graft material for fusing the first bone structure to the second bone structure;
an insertion tool interface configured to secure the implant device to an insertion tool; and
a fixation interface including a plurality of screw alignment holes, wherein each of the plurality of screw alignment holes is configured to receive a screw and to orient the screw at a predetermined angle, wherein each of the plurality of screw alignment holes defines a cavity configured to house a head of the screw within the implant device body after insertion of the implant device is complete.

15. The implant device of claim 14, wherein the predetermined angle is configured to cause a tip of the screw to extend through the upper surface of the implant device body or the lower surface of the implant device body and into a bone structure adjacent to the implant device body after insertion of the implant device body is complete.

16. The implant device of claim 14, wherein each of the plurality of screw alignment holes includes a locking ring configured to prevent rotation of the screw after insertion of the implant device body is complete.

17. The implant device of claim 14, wherein the plurality of screw alignment holes includes a first alignment screw hole configured to orient a first screw at a first predetermined angle to cause a tip of the first screw to extend through the upper surface of the implant device body and into the first bone structure after insertion of the implant device body is complete, and a second alignment screw hole configured to orient a second screw at a second predetermined angle to cause a tip of the second screw to extend through the lower surface of the implant device and into the second bone structure after insertion of the implant device body is complete.

18. The implant device of claim 17, wherein the plurality of screw alignment holes includes a third screw alignment hole configured to orient a third screw at the first predetermined angle or the second predetermined angle to cause a tip of the third screw to extend through the upper surface of the implant device body and into the first bone structure after insertion of the implant device body is complete or to extend through the lower surface of the implant device and into the second bone structure after insertion of the implant device body is complete.

19. The implant device of claim 14, wherein the insertion tool interface includes a plurality of apertures configured to secure the implant device to an insertion tool, wherein the plurality of apertures are accessible from at least one edge of the one or more edges of the implant device body and extend into the implant device body, wherein a first aperture of the plurality of apertures is a threaded aperture configured to rotatably secure the implant device body to a threaded element of the insertion tool, and wherein a second aperture of the plurality of apertures is configured to receive a finger element of the insertion tool, and wherein the at least one edge of the implant device body is an anterior edge of the implant device, and wherein the plurality of apertures extend into the implant device body from the anterior edge towards a posterior edge of the implant device.

20. The implant device of claim 14, wherein the insertion tool interface includes a plurality of apertures configured to secure the implant device to an insertion tool, wherein the plurality of apertures are accessible from at least one edge of the one or more edges of the implant device body and extend into the implant device body, wherein a first aperture of the plurality of apertures is a threaded aperture configured to rotatably secure the implant device body to a threaded element of the insertion tool, and wherein a second aperture of the plurality of apertures is configured to receive a finger element of the insertion tool, and wherein the at least one edge of the implant device body is a lateral edge of the implant device, and wherein the plurality of apertures extend into the implant device body and away from the lateral edge of the implant device.

21. The implant device of claim 19, wherein the plurality of apertures includes a third aperture configured to receive a second finger element of the insertion tool, wherein the threaded aperture is disposed within the insertion tool interface between the second aperture and the third aperture, and wherein the second aperture and the third aperture extend into the implant device body in a direction that is parallel to a longitudinal axis of the threaded aperture, a direction that is angled towards the longitudinal axis of the threaded aperture, a direction that is angled away from the longitudinal axis of the threaded aperture, or a combination thereof.

22. An implant device for insertion between a first bone structure and a second bone structure, the implant device comprising:
an implant device body including an exterior surface, wherein the exterior surface of the implant device body includes an upper surface defining a top of the implant device, a lower surface defining a bottom of the implant device, and one or more edges defining a portion of the exterior surface between the upper surface and the lower surface, wherein the implant body has a thickness defined by the upper surface and the lower surface, and wherein the implant body defines a plurality of receptacles configured to receive graft material for fusing the first bone structure to the second bone structure;
an insertion tool interface including a plurality of apertures configured to secure the implant device to an insertion tool, wherein the plurality of apertures are accessible from at least one edge of the one or more edges of the implant device body and extend into the implant device body, wherein a first aperture of the plurality of apertures is a threaded aperture configured to secure the implant device body to a threaded element of the insertion tool, and wherein a second aperture of the plurality of apertures is configured to receive a finger element of the insertion tool; and
a fixation interface including a plurality of screw alignment holes, wherein each of the plurality of screw alignment holes is configured to receive a screw and to orient the screw at a predetermined angle, wherein each of the plurality of screw alignment holes defines a cavity configured to house a head of the screw within the implant device body after insertion of the implant device is complete.

23. The implant device of claim 22, wherein each of the plurality of screw alignment holes includes a locking ring and a groove, wherein the locking ring includes a c-clip and threads configured to rotatably receive the screw, wherein tightening of the screw causes the c-clip to enter the groove, and wherein the locking ring prevents the screw from backing out of the screw alignment hole after insertion of the implant device body is complete.
